(19) United States
(12) Patent Application Publication
(10) Pub. No.: US 2011/0177169 A1
(43) Pub. Date: Jul. 21, 2011

(54) OPHTHALMIC FORMULATIONS OF REVERSED LIQUID CRYSTALLINE PHASE MATERIALS AND METHODS OF USING

(76) Inventors: David Anderson, Ashland, VA (US); Vince Conklin, Glen Allen, VA (US); Benjamin Cameransi, Georgetown, SC (US); David M. Kleinman, Rochester, NY (US); Eugene R. Cooper, Berwyn, PA (US)

(21) Appl. No.: 12/833,516

(22) Filed: Jul. 9, 2010

Related U.S. Application Data
(63) Continuation-in-part of application No. 12/731,901, filed on Mar. 25, 2010.
Application No. 12/731,901 is a continuation of application No. 10/889,313, filed on Jul. 13, 2004, now Pat. No. 7,713,440.
(60) Provisional application No. 61/224,712, filed on Jul. 10, 2009.

(51) Int. Cl. A61K 9/10; A61K 9/00; A61K 38/13; A61K 39/00; A61K 31/573; A61K 31/445; A61K 39/395; A61K 31/685; A61K 31/355; A61K 9/14; A61P 37/06; A61P 29/00; A61P 27/02; B82Y 5/00

(52) U.S. Cl. 424/484; 424/400; 514/20.5; 424/184.1; 514/180; 514/330; 424/141.1; 514/78; 514/458; 424/489; 977/906

(57) ABSTRACT

The eye is effectively treated by providing it with formulations including uncoated cationically charged microparticles of reversed cubic phase or reversed hexagonal phase material. The treatment methods are effective; for a variety of diseases and conditions including dry eye. The structure, charge and components of the microparticles in dispersion, with or without an active ingredient, provide mucoadhesion, layering, protection and prolonged duration of ophthalmic action.

### CROSS-REFERENCE TO RELATED APPLICATIONS

This application claims priority to U.S. patent application Ser. No. 61/224,712 “Cationic Particles of Reversed Liquid Crystalline Phase Materials and Methods of Using”, filed Jul. 10, 2009, and the complete contents thereof is herein incorporated by reference. This application is also continuation-in-part (CIR) of U.S. patent application Ser. No. 12/731,901 filed Mar. 25, 2010, which is a continuation of U.S. patent application Ser. No. 10/889,313, filed Jul. 13, 2004, now U.S. Pat. No. 7,713,440 “Stabilized Uncoated Particles of Reversed Liquid Crystalline Phase Materials” issued May 11, 2010, and U.S. Patent Application Ser. No. 60/509,255, filed Oct. 8, 2003, and this application claims priority to each of these applications and herein incorporates them by reference.

### BACKGROUND OF THE INVENTION

1. Field of the Invention

The invention generally relates to uncoated particles of reversed cubic phase or reversed hexagonal phase material containing a cationic bilayer-associated compound, and having a cationic charge causing substantial binding to, or other attractive interaction with, compounds and tissues of the body, enabling improved efficacious treatment of various conditions and diseases, especially of the eye.

2. Description of the Prior Art

Nanostructured lyotropic liquid crystalline phases of the reversed type-namely reversed cubic and reversed hexagonal phases-have been developed as excellent solubilizing matrices for both poorly soluble compounds, and for such delicate compounds as proteins and biomacromolecules. U.S. Pat. No. 6,482,517 (Anderson, Nov. 19, 2002) and U.S. Pat. No. 6,638,621 (Anderson, Oct. 28, 2003) the contents of which are herein incorporated by reference, disclose, among other things, effective compositions and methods for producing such lyotropic liquid crystalline matrices. U.S. Pat. No. 5,531,925 to Landh, Jul. 2, 1996, describes particles of reversed cubic or reversed hexagonal phase which require a surface phase of either lamellar liquid crystalline, lamellar crystalline or L3 phase.

### SUMMARY

This Application is an improvement on the prior art on the use of positively charged particles in drug delivery and specifically ophthalmic drug delivery and treatment of dry eye. These approaches, such as in U.S. Pat. No. 6,007,826 and publicized by NovaGali Pharma are typically based upon emulsions. In contrast, this Application describes a synergistic combination of charge, structure and materials.

In U.S. Pat. No. 7,713,440, priority to which is claimed above, it is taught that particles of reversed lyotropic liquid crystalline phase materials can exhibit high potential to transport active compounds across a variety of barriers such as cell membranes, particularly in the case of the reversed bicontinuous cubic phases, by virtue of their unique nanoporous structures and associated curvature properties. The reversed cubic and reversed hexagonal liquid crystalline phases can be of very low solubility in water, meaning that they maintain their integrity as vehicles upon entry into the body thus avoiding drug precipitation. Ionically charged, bilayer-associated compounds with appropriate chemistries and concentrations can stabilize such particles as uncoated particles by creating strong electrostatic surface potentials˜particle zeta potentials and assist in binding. In particular, zeta potential is a key parameter for establishing such stabilization as well as good attraction to mucins and adhesion to mucosal surfaces and other tissues, and in particular that a zeta potential of greater than or equal to about +25 mV, or more preferably greater than about +30 mV, in magnitude is an important requirement for such a system. For ophthalmic formulations, this must be achieved with compounds that are non-irritating to the eye. U.S. Pat. No. 7,713,440 thus describes stable, uncoated particles formed of reversed lyotropic liquid crystalline materials. The particles are “uncoated” in that the liquid crystalline material of which the particles are formed is in direct contact with the medium in which the particles are dispersed, i.e. the outer periphery of an individual, dispersed particle is not shielded from the medium (for example, an aqueous liquid phase) in which the particles are dispersed. No coating intervenes between the particle and the medium, or between a particle and other particles. Rather, the particles are repelled from one another and are held in dispersion in the medium by strong electrostatic surface potentials. Such strong electrostatic surface potentials are created by proper choice of the “ingredients” which are combined to make up the liquid crystalline material of which the particles are formed.

Cationic emulsions for dry eye treatment are exemplified by the approach of Benita and Lambert in U.S. Pat. No. 6,656,460. Such an emulsion comprises a non-polar phospholipid, a non-polar oil, a non-toxic emulsifying agent, and a cationic lipid which imparts to the emulsion a net positive charge. The non-polar oil is typically a triglyceride. This patent relies on non-polar oils for emulsion formulation, although it points out in test eye treatments, triglycerides (which are non-polar oils) were cited as having local toxic effects in eye preparations, in particular “ . . . olive oil, rather than CsA was responsible for the surface epithelial defects developing in the cornea.” Although referred to as “non-toxic”, the emulsifying agents also required in the approach of Benita and Lambert, typified by Poloxamer 188, are non-biodegradable detergents and can have/both local and systemic toxicity, as well as mitigating effects on the attractive droplet-tissue interactions that are central to that patent. The emulsion compositions of Benita and Lambert and others, are radically different than reversed cubic and reverse hexagonal phase lyotropic liquid crystalline material, with starkly different structure, function, behavior and impact in the pharmaceutical setting.

The surface of the eye is covered by a complex tear film. The tear film of the eye is comprised of three layers, which serve multiple functions including the following. The outer oily layer is produced by meibonian glands in the eyelids, and reduces evaporation. The middle, thick, watery layer is made by the lacrimal gland above the upper eyelid and is thought to wash away irritants. The inner mucus layer is secreted by goblet cells in the conjunctiva of eyelids and helps the tear film stick to the corneal surface.

In the instant Application, the cationically charged uncoated reversed cubic or reversed hexagonal liquid crystalline material particle formulations and methods of use developed have surprising and novel features and benefits for therapy, including but not limited to drug delivery, and including ophthalmic and dry eye treatments, relating to novel synergies created in attraction, adherence, coating, permeability, diffusion and delivery.

It is an object of this invention to provide

Cationically charged lipidic particles (ranging in size, depending upon the materials and the processing selected for the intended application, from under 100 nanometers to 1 micron in diameter, and in some instances larger—as an example, the cationically charged lipid particles may have size ranging from 10 nm to 100 microns, although, in generally smaller sized particles would be preferred) (hereinafter simply called particles or microparticles) exhibiting strong attraction and adhesion to certain tissues and compounds, such as those at the surface of the eye, including mucins, to enhance the delivery of drugs, nutrients and other compounds, and to afford protective coating over these tissues and, in the case of the eye, the associated tear film. The particles are able to solubilize active pharmaceutical compounds at concentrations at least as high as those achievable in emulsions, and play a supportive role in absorption of the active compound by the appropriate cells. Being lipid-based and mucoadhesive, the microparticles can, for example, lay down a film of lipid over the tear film or over the ocular tissue for the purpose of alleviating the symptoms of dry eye and related conditions, and if loaded or delivered with active drug can furthermore provide effective delivery of the active to the appropriate ocular tissue. The cationic charge of the particles increases the attraction to and interaction with mucin and cells of the surface of the eye, as well as enhances the dispersion and distribution of the particles on the tear film after application to the eye by electrostatic repulsion of one another because of similar charge. Because the particles are formed of a matrix material with simultaneous continuity of both hydrophobic (bilayer) and hydrophilic (aqueous pore) networks, in particular with continuous aqueous channels permeating the material including each particle, and because the particles are uncoated, these channels are in hydraulic continuity with the aqueous media surrounding the particle (that is, they are accessible pores), the latter media consisting of extra-or intra-cellular fluids or in the particular case of opthmalmic applications, the tear film of the eye.

Particles which have distinct and substantial advantages over other lipid vehicles for drug delivery and therapy (principally liposomes and emulsions) including to the eye. The particles described herein have a distinct structure from emulsions, microemulsions, liposomes, micelles, and other particles, and behave preferably at the cellular interface, and in the presence of the tear film at the corneal surface. Because of the presence of continuous aqueous channels (accessible pores), such a particle allows the aqueous phase within and outside the particle to interchange material, even when the particles form a continuous layer, whether at the surface of the eye tissue or at the tear film/air interface.

Particles which are preferably free from ingredients, such as triglycerides, in amounts that necessitate the addition of preservatives in amounts that can be irritating to the eye.

Particles which, in one embodiment, are prepared without an active pharmaceutical ingredient (API) in the traditional pharmaceutical sense, but with components that are known to have biological activity relevant to therapy to the eye, including the treatment of dry eye, such as phosphatidylcholine, glycerol, and vitamin E, and are acceptable for over the counter (OTC) use.

Administrable drug-loaded or active-loaded microparticles, in another embodiment, that take full advantage of the absorption-promoting and drug-solubilizing potential of reversed cubic and reversed hexagonal liquid crystalline phase microparticles, particularly bicontinuous phase, undiminished by effects of coatings, and enhanced by cationic charge. In some embodiments of the invention, a therapeutic compound, typically though not always a drug substance, is dissolved or dispersed or otherwise incorporated within the liquid crystalline phase material itself. Preferably, in this embodiment, the therapeutic compound is solubilized within the liquid crystalline material, making it an integral part of the liquid crystal. One advantage of such a particle is that the therapeutic compound reaps the benefit of the absorption-promoting capabilities of the liquid crystal, in a manner that is superior to particle configurations described elsewhere, where the compound is present primarily outside the liquid crystal, or inside a liquid crystal particle that is covered with an interfering coating. Indeed, it is envisioned that in many cases, the majority of therapeutic compounds will remain associated with the liquid crystal up to the point where the liquid crystal integrates with, for example, a targeted cell membrane, thereby eliminating the need for the compound to dissolve in an aqueous biological fluid (e.g., blood, intestinal fluid) en route to cellular uptake. It is also of major impact herein that this can all be accomplished within the context, and extreme restrictions, of ophthalmic formulations, where the potential for counterproductive or even dangerous irritation exists, as well as other mucous membranes, and in injectable formulations including intravenous pharmaceutical formulations.

Administrable drug-loaded microparticles that exhibit direct, unhindered interactions with biomembranes which can strongly promote absorption, allow targeting and extend covering, particularly to mucosal and other anionically-charged tissues or cells throughout the body, including but not limited to nasal, oral, respiratory, intestinal, and vaginal.

Stable dispersions of such drug-loaded lyotropic liquid crystalline microparticles for ophthalmic application.

A method for treating a mammal with a pharmaceutical or nutriceutical compound by administering a dispersion of cationically charged uncoated particles of reversed liquid crystalline phase material.

Compositions that yield charge-stabilized particles and dispersions thereof upon reconstitution with water or other hydrophilic milieu, including natural or artificial human tears.

New compositions and methods for the delivery of compounds that are of use and efficacy in the treatment of dry eye and other conditions of the eye, including such active pharmaceutical ingredients (APIs) as cyclosporine, dexamethasone, and triamcinolone, and treatment of dry eye without requiring a “traditional” API, as in the case of OTC products.

Cationic microparticles that bind nucleic acid compounds such as DNA, RNA, RNAi, antisense compounds, plasmids, and other related compounds by virtue of the attractive interaction between the cationic microparticle and the anionic nucleic acid compound.

Cationic microparticles that disperse and form a coating on or at the tear film/air interface, preventing the break up of the tear film, protecting and reducing evaporation of the tear film, or on the tissue of the eye, protecting and providing prolonged residence time and enabling prolonged and sustained protection and delivery of actives.

Cationic microparticles comprised of an accessible aqueous phase allowing water and aqueous compounds, including but not limited to drugs, proteins and electrolytes, to enter and exit such particles.

Cationic microparticles made of a wide range of lipids, surfactants, fats, fatty acids and other components, among which are compounds found in or beneficial to the eye, including the tear film.

Cationic microparticles that are effective delivery vehicles for proteins, in particular the proteins for therapy of conditions of the eye, including the tear film.

Cationic microparticles that are effective delivery vehicles for proteins, in particular the proteins for therapy of conditions of the eye, including the tear film.

Cationic microparticles that may be adapted to absorb one or more materials from the environment of the eye, or to release one or more materials, such as therapeutic agents, into such environment.

Cationic microparticles that can be used, with or without an Active Pharmaceutical Ingredient (API), to lay down a beneficial lipid-rich coating on a tissue or a liquid film, including the eye and the tear film. The coating, due to the bicontinuous channels comprising the reverse cubic and reverse hexagonal phase uncoated particles, allows flow of aqueous phase and its contents from one side of the coating, through the particles, to the other side. The aqueous pores would retain natural tears and water soluble compounds. The coating would not obstruct the circulation of aqueous or water soluble compounds from one side of the coating to the other, or between the interior of the particles and outside of the particles. Such a film, as established by either components of the particles or by the particles themselves (or fragments thereof), can apply in one or more areas or interfaces including, but not limited to, most preferably, the corneal surface, the mucin-rich region of the tear film, and/or the interface between the tear film and air. By lowering the surface tension of the tear film, and/or by improving the wettability of the corneal surface (for example, by deposition of polar lipid-rich material with polar moieties of the lipid establishing a more hydrophilic milieu), the integrity of the tear film can be improved and its break-up time increased substantially. Dwell time can be increased. Evaporation can be decreased. Corneal residence time can be prolonged. Such particles protect the eye while allowing diffusion and delivery of water soluble actives, nutrients and other materials. The particles would enhance or improve upon the lipid coating on the tear film, or if such coating were not present, take the place thereof.

### DETAILED DESCRIPTION

In this invention, drug-loaded cationically charged microparticles of reversed cubic and reversed hexagonal liquid crystalline phases are dispersed in a liquid such as water, without any coating, thus permitting direct interactions between the liquid crystal and biological barriers. In addition, this can be achieved using only components that are pharmaceutically-acceptable for intravenous injection and particularly for topical ophthalmic application which is a necessary requirement for the use of such dispersions in certain critical drug-delivery applications. This is achieved in the present invention by compositions containing cationically charged, bilayer-associated components that yield an electrostatic potential on the particles sufficiently strong to stabilize the particle dispersions against aggregation, flocculation, and fusion; generally, this requires a zeta potential greater than or equal to about +25 mV, or more preferably greater than about +30 mV. The cationic charge is also used to enhance attraction and binding of the particles to anionically charged tissue and cellular targets, allowing intimate association of the particle and such surfaces while the particle, comprised of continuous aqueous channels, does not prevent diffusion of water and water-soluble materials into and out of the particle.

In a dispersion of uncoated particles according to the present invention the drug (or more generally, active, or therapeutic compound) (in embodiments where a drug is employed) is present in the reversed cubic phase or reversed hexagonal liquid crystalline phase particle. In the most preferred embodiment, the drug or active is a component of the reversed cubic phase or reversed hexagonal phase. In alternative embodiments, the drug or active is dissolved or dispersed or embedded or otherwise incorporated within the particle. In one variation, the drug or active may be incorporated in an oil phase that is positioned within the particle. (Cf. U.S. Pat. No. 6,991,809, the contents of which are incorporated in their entirety by way of reference).

A schematic of the electrostatic configuration in a representative uncoated particle of the instant invention would show a net positive (cationic) surface ionic charge. The cationic moieties, represented by plus signs, would include the charged, bilayer-associated compounds utilized in the invention. As one moves away from the (cationically-charged) surface of the particle, the preponderance of positive charges diminishes. The zeta potential measurement measures the potential due to the excess of ionic charges (in this case, anionic) at the shear plane, which is displaced from the particle surface. Nevertheless, at least in the conditions used in the Examples below and quite broadly in the practice of this invention, the shear plane still lies within the Debye layer, which extends a distance (the Debye length) out from the particle surface where there is no longer a net excess of anions.

As used herein, an uncoated particle of reversed cubic (or hexagonal) phase is a particle in which the outermost material phase of the particle is a reversed cubic (or hexagonal) phase, so that there is no other phase present exterior to and in contact with this outermost material phase except for a single liquid (usually aqueous) phase in which the particles are dispersed (dispersion phase), and wherein the material of this reversed cubic [hexagonal] phase is a single, contiguous and isolated mass of material thus defining a single particle. In this definition “isolated” means substantially not in contact with other such particles except for the normal particle-particle collisions in the course of Brownian motion The uncoated particle thus defined contrasts with the particles described U.S. Pat. No. 6,482,517 in which there is a crystalline coating exterior to the liquid crystalline phase, and also in contrast with U.S. Pat. No. 5,531,925 and the work of P. A. Winsor cited above in which there is a distinct L3 phase, lamellar phase, or crystalline lamellar phase exterior to (i.e. coating) the reversed liquid crystalline phase. As discussed herein, the L3 and lamellar coatings in particular are antithetical to the purpose of employing the particles in permeability enhancement for improved drug delivery, and they may furthermore introduce other limitations and practical problems.

A compound or moiety is bilayer-associated if it partitions preferentially into a bilayer over an aqueous compartment. Thus, if a bilayer-rich material such as a reversed cubic phase material exists in equilibrium with excess water and is placed in contact with excess water, and a bilayer-associated compound or moiety is allowed to equilibrate between the two phases, then the overwhelming majority of the compound or moiety will be located in the bilayer-rich phase. The concentration of the compound or moiety in the bilayer-rich phase will be at least about 100 times, and preferably at least about 1,000 times, larger than in the water phase.

For the purposes of this disclosure, for brevity the term “stabilized particle” will mean a particle that can, in plurality, form a stable dispersion in a liquid, preferably a liquid comprising a polar solvent, and most preferably comprising water or glycerol. A stable dispersion means that the particle dispersion does not show detrimental effects from flocculation or fusion over timescales of at least several days, preferably several weeks and most preferably over several months or more.

A targeting moiety is a chemical group that is part of the particle of the instant invention, situated either inside the liquid crystal or bound to the surface of the particle, and serves as a molecular target for some compound outside the particle in the application, typically though not always a biomolecule in the body of a mammal. A targeting compound, then, is a compound that contains a targeting moiety. It is important to point out that the targeting moiety is incorporated in the current invention without the introduction of another phase (a “coating” phase, or “dispersing” phase) at the surface of the particle.

Particles of the instant invention may be superior in treating disorders of the eye to two lipid-based vehicles from the prior art, most notably liposomes and oil/water emulsions, by virtue of one or more of the following:

A) In contrast with liposomes and emulsion droplets, a particle of the instant invention which is based on reversed bicontinuous cubic phase material is laced throughout with a continuous network of aqueous pores, that is, it is water-continuous, with the interior pores being accessible from the exterior aqueous phase. This means that if a contiguous layer or bed of particles is laid down, such as on a tissue surface or a tear film, then regardless of the degree of coverage, the particles will not form a fully occluding layer; and at degrees of coverage anticipated for a clinical or patient-use situation, the layer formed by the particles will allow for facile permeability of water and water-soluble substances, including nutrients, drugs or other bioactive compounds, through the layer. Indeed, based on diffusion calculations by the inventor for aqueous materials within cubic phases, the effective self-diffusion coefficient of a water-soluble compound within the layer will be at least one-tenth of, and more preferably at least one-third of, the self-diffusion coefficient of the compound in pure aqueous buffer. This contrasts with emulsion droplets, which do not have aqueous pores, and with liposomes, which contain water in their core but in which said water is not accessible from the exterior aqueous phase. These latter lipidic particles are capable of forming layers that occlude the underlying tissue (cornea, etc.), particularly since the particles are capable of deforming, and/or releasing interior components to leave behind residual lipid films (phospholipid-rich, and/or triglyceride-rich); indeed, in both liposomes and emulsions, the compositions are deliberately, or tacitly, chosen to promote the formation of lamellar liquid crystalline or lamellar crystalline materials, which are in turn prone to forming occluding layers known to be largely impermeable to aqueous solutes. By contrast, in the instant invention, one or more water-soluble drugs could, for example, be incorporated into the exterior aqueous phase without the risk of poor delivery engendered by occluding vehicles.

B) The same system of accessible aqueous pores can also provide for deposition of water-soluble nutrients or other bioactive compounds onto the tissue surface, e.g., the corneal epithelium, and promote retention of these same compounds even in the face of desiccation, tear-film breakup, or the sweeping action of the eyelid during blinking, etc.

Such compounds include peptides and particularly proteins, which are typically soluble in the phospholipid-based reversed liquid crystalline phases that are the basis of the preferred embodiments of this invention.

C) By virtue of simultaneous continuity and accessibility of aqueous domains throughout particles of this invention, water-soluble bioactive compounds, including proteins, inside the particles are far more accessible to the eye than in the case where they are entrapped in a liposome, for example. By contrast, the aqueous core of a liposome is not accessible. Emulsion droplets, in contrast, contain no aqueous core and are poorly suited for solubilization of proteins without denaturation.

D) In the event that nerve endings are present at, or near, the surface at which particles of this invention bind, then the aspects mentioned in A), B) and C) above could provide relief from irritation, as nerve tissue would likewise be relatively protected and yet have access to nutrients and other important bioactive compounds. Particles of the invention could be useful in treating neurogenic dry eye, and in reducing inflammation due to dry eye (with, or even without, the incorporation of a therapeutic amount of anti-inflammatory drug in the particles).

E) The same system of accessible pores, which also have a cationic surface charge, throughout the interior of the particles of the invention can provide for a high capacity of particle-bound mucin, meaning that the particles can be very effective in carrying, depositing or capturing soluble mucins—which might otherwise be in the interior of the tear film at a distance from the corneal surface—onto the corneal epithelium or other tissue. This could bring down mucins onto patches of the eye that would otherwise be mucin-lean. The volume fraction of accessible, cationic pores in the particles of this invention is preferably at least 20%, and more preferably at least 30%. The extra degree of mucin-binding due to the presence of an accessible cationic-walled pore space, unique to this invention, can also increase the effectiveness of another possible therapeutic aspect of cationic particles, namely their ability to bind simultaneously to soluble (unbound) and bound mucins, effectively binding the former to the eye surface. It can be added that, due to the well-known increase in strength of binding as the number of attachment points increases, the accessible cationic pores can provide for much stronger binding of particles of the instant invention. By bringing more mucins to the surface of the eye, the wettability of the ocular surface will improve, thus allowing more even aqueous tear layer on the eye. Particles of the invention could be used to treat mucin deficiency, by effectively turning unbound mucins into bound mucins, which has the additional effect of reducing unbound mucins and thus, potentially at least, triggering increased mucin production.

F) The aqueous fraction of a reversed liquid crystalline phase material is typically strongly bound within the pores, and thus less prone to evaporative loss than is water in larger pools, due to a combination of several factors, such as the nanometer-scale diameter of the pores (and associated capillarity effects), the close association with lipid head groups, etc. Thus, particles of the present invention constitute an accessible reservoir of water bound to the cornea or otherwise present in the eye, and thereby provide relief from desiccation even after tear-film breakup. As in the case of nutrient/protein transport discussed in the above points, accessibility of the water in these pores to the greater tear film, by virtue of the hydraulic continuity between the water in these pores and the water outside the particles, is of crucial importance, and is not in effect for certain other lipidic particles such as liposomes. The dispersion, by nature of its lipid structure and charged surface will also distribute on the surface of the tear film at the tear/air interface. The lipid structures can then serve to decrease aqueous evaporation and prolonging the time that aqueous tears remain distributed across the eye. Such an effect will also promote ocular health and provide relief from dry eye, especially when associated with lipid deficiency and decreased tear break up time.

G) Since the particles of the instant invention are able to solubilize a wide range of proteins and other bioactive compounds, with a high loading capacity due to the huge bilayer surface area, and with such compounds accessible due to the accessibility of the pores and lipid-lined pore wall surface, they are excellent for bathing, protecting, and replenishing regions of the eye surface. Phrased otherwise, layers of phospholipid-based particles of this invention, potentially containing other nutrients or protectants, including proteins, can act as (semi)artificial epithelial surfaces, with composition selected for optimization of the outermost surface. As an example, the incorporation of a PEGylated phospholipid (e.g., N-(carbamoyl-methoxy-polyethyleneglycol 2000)-1,2-distearoylphosphatidylethanolamine, or “PEGylated-PE”) or other PEGylated lipid can provide a layer that has fouling-resistant and water-binding properties known to make PEG compounds so useful in treating surfaces which are in contact with biological fluids. A corneal surface treated with particles of the invention comprising such an adsorption-deterring component could reduce corneal accumulation of materials detrimental to the eye, such as endogenous inflammatory mediators or infection-related endo- or exotoxins.

H) Goblet cells are a main secretor of soluble mucins, and their numbers fall in those suffering from dry eye. Via one or more of the mechanisms discussed herein by which particles of the invention can improve the health of mucosal tissue, the particles could also play a role in improving goblet cell health, further enhancing the benefit to the dry eye patient. By keeping goblet cells from desiccating, as discussed herein with regard to the accessible aqueous channels and water-binding polar lipids of particles of the invention, this can enhance mucin production. Furthermore, there is peptide signaling promoting goblet cell mucin secretion, so it is an important advantage that the aqueous pores of particles of this invention may maintain access of these peptides to the goblet cells even as it covers them, while keeping them moist, thus enhancing goblet cell survival and also promoting a healthy tear layer. If the peptides diffuse into the accessible pores of the particles and are thus substantially protected from degradation while in the pores, this may prolong their half-life and thus help normalize corneal health. Also by protecting corneal nerves as discussed herein, these nerves can maintain more normal function and better maintain their ability to release neurotransmitters and growth factors to stimulate goblet cell mucin secretion. By increasing the quality of the tear film and through the aqueous pores, goblet cells could have improved access of the secreted proteins, growth factors, and peptides.

(I) It is important to point out that, while the particles of the instant invention—by virtue of a number of features including cationic charge and the associated effect on attraction, binding and dispersion, high drug loading capacities, accessibility of both interior lipid and aqueous domains—can be superior entities for the delivery of drug substances to the eye or other mucinous, or non-mucinous, tissue, in certain application such as dry eye the particles may be quite effective therapeutically even in the absence of an explicit or traditional drug substance. The terns “API” and “drug substance” engender various definitions, connotations and expectations and these can be dependent on whether one is in a pharmaceutical, ophthalmic, regulatory, or patent setting. In any case, an important embodiment of this invention is typified by certain compositions in the Examples section wherein the totality of the components, namely phosphatidylcholine-rich lecithin, alpha-tocopherol, oleylamine, water, glycerol and mannitol, do not include any compound that is considered a “drug”, “drug substance”, or “API” in the narrowest pharmaceutical sense of these terms, and wherein the therapeutic benefit of the composition is as much due to the structure and surface chemistry (especially charge) of the particles, than to any particular “active” ingredient. Thus they may be used to form compounds which fall under the FDA's OTC regulations. Thus, for example, although the FDA technically considers glycerol to be an “active” in ophthalmic formulations for dry eye, it's function is clearly more “mechanical”, as a lubricant and softening fluid, and it does not function at the microscopic level of the cell via interaction with individual target molecules (usually receptors, enzymes, or nucleic acids) as does the traditional “drug substance”. Further, to refer to, for example, oleylamine as an “API” in some of the Examples below would be to miss the point that oleylamine alone by itself has little if any therapeutic effect in the doses intended, in contrast with, say, a steroidal anti-inflammatory or cyclosporine (e.g., Example 14). Rather, the reversed liquid crystalline particles comprised of these compounds and disclosed herein, by virtue of their cationic charge and other physical characteristics, are prone to bind strongly to the corneal surface, and form a protective layer that, as described above, provides a natural phosphatidylcholine-rich biomimetic layer which is nevertheless non-occluding and able to give rise to a local enrichment of mucins, water, lipids, and various endogenous compounds. In the case of dry eye, the inventor notes that in certain therapeutic approaches, all the materials needed to provide relief from or treatment of the condition are present in the eye, and the function of the particles is to sequester and maintain these materials over the eye surface in a way that effects therapy. Thus, no explicit “drug substance” is needed for many of the foreseen applications of this invention. For the purposes of this disclosure, the terms “active” and “therapeutic compound” are taken to be broader and more inclusive than the terms “Active Pharmaceutical Ingredient”, “drug”, and “drug substance”.

J). Particles of the instant invention can be made with materials that do not readily lend themselves to incorporation into alternative delivery vehicles, such as liposomes or emulsions. In some embodiments, this allows particles of the instant invention to avoid materials which are undesirable for the application, such as, for example, the use of triglycerides, which tend to promote rapid spoilage by microbes, in certain ophthalmic applications. While traces of triglycerides may be present in such components as phospholipids or bile salts, certain embodiments of the instant invention will preferably have triglyceride concentrations less than about 1 mg/mL, more preferably less than about 0.1 mg/mL, and most preferably less than about 0.01 mg/mL. All of the Examples below have triglyceride concentrations less than 0.1 mg/mL, and many of the Examples (such as Example 2) have triglyceride levels less than 0.01 wt/wt %. In other embodiments, this permits the use of a material which brings particular benefit to the application, where that is practically unachievable with, for example, liposomal technology.

The foregoing advantages may apply to tissues, cells and targets in the body other than in the eye. Nasal, oral and vaginal are examples of mucinous tissues particularly amenable to treatment with the present invention.

### EXAMPLES

### Methods and Materials

The Methods and Materials section of U.S. Pat. No. 7,713,440 describes methods and materials for making uncoated charge stabilized particles in general, and these methods and materials are preferably used in the practice of this invention. The Examples describe particular embodiments of the particles, which are illustrative of the invention, but from which one of ordinary skill in the art could readily prepare alternative formulations within the spirit and scope of the claimed invention. Certain aspects of this invention are herein further disclosed.

After, or concomitantly with, the selection of liquid crystal composition, one or more appropriate ionically-charged, bilayer-associated components is/are selected based on such properties as partition coefficient (generally high is best, preferably greater than about 1,000), low toxicity, favorable regulatory status (dependent on the route of administration), and solubility and compatibility with the other components of the formulation. The incorporation of a cationically-charged, bilayer-associated compound that induces a charge throughout the bilayer, and creates a surface charge on particles of the liquid crystalline material is a key aspect of the invention. There are two general methods for incorporating this charged compound, although the net result is typically not affected by the choice of method. In one method, the charged compound is mixed together with the liquid crystalline material—or in some cases, the reversed liquid crystalline phase requires the presence of the charged compound. In another method, the charged compound is present in the liquid phase, preferably solubilized therein, and the liquid crystal is dispersed in this mixture. In the end, the components will tend toward equilibration, which will tend to minimize the difference between these approaches, such that the charged component will partition between the liquid crystalline particles and the polar phase according to a distribution that eventually would come to an equilibrium, or near-equilibrium, distribution.

The positively charged, bilayer-associated compound will often, though not always, be a cationic surfactant. Examples of such surfactants, pharmaceutically-acceptable for various routes of administration, are given below. In many embodiments of the invention, however, the charged compound will not satisfy the definition (given above) of a surfactant, but will nonetheless be perfectly well suited as a charged, bilayer-associated compound capable of yielding particles of the instant invention. The charged bilayer associated compound may be the active, e.g., an API.

Preferred cationic, bilayer-associated compounds include: stearylamine, oleylamine, benzalkonium chorlide, tocopheryl dimethylaminoacetate hydrochloride, Cytofectin gs, 1,2-dioleoyl-sn-glycero-3-trimethylammonium-propane, cholesterol linked to lysinamide or ornithinamide, dimethyldioctadecyl ammonium bromide, 1,2-dioleoylrsn-3-ethylphosphocholine and other double-chained lipids with a cationic charge carried by a phosphorus or arsenic atom, trimethyl aminoethane carbamoyl cholesterol iodide, O,O′-ditetradecanoyl-N-(alpha-trimethyl ammonioacetyl)diethanolamine chloride (DC-6-14), N-[(1-(2,3-dioleyloxy)propyl)]-N-N-N-trimethylammonium chloride, N-methyl-4-(dioleyl)methylpyridinium chloride (“saint-2”), lipidic glycosides with amino alkyl pendent groups, 1,2-dimyristyloxypropyl-3-dimethylhydroxyethyl ammonium bromide, bis[2-(11-phenoxyundecanoate)ethyl]-dimethylammonium bromide; N-hexadecyl-N-10-[O-(4-acetoxy)-phenylundecanoate]ethyl-dimethylammonium bromide, 3-beta-[N—(N′,N′-dimethylaminoethane)-carbamoyl. Peptides rich in lysine, arginine, and histidine, when used at pH's less than their pI (which can be quite high), are useful provided they partition preferentially into the reversed liquid crystalline phase over water. It should be noted that lysozyme does not partition into typical cubic phases, as typified, e.g., by the cubic phases used in the Examples below.

In the course of this work it was established that once the zeta potential of a collection of these reversed liquid crystalline phase particles equals or exceeds about +25 millivolts, or preferably greater than about +30 mV, then no other mechanism is required for stabilization of the dispersion against flocculation. In some cases, other exceptional attractive forces, such as intermingling of surface-associated polymer chains, unusual ionic conditions, time-dependent redistributions within the particles, may prevent the formation of stabilized particles by this method alone.

Some particles of use in this invention may be cationically charged sufficient to enhance attraction and delivery as herein described, but insufficient in and of itself to stabilize the particles absent an additional force, such as steric stabilization.

Lipids and surfactants form the basis of the lyotropic liquid crystalline phases that are a fundamental building block of the current invention and preferred surfactants are set forth in U.S. Pat. No. 7,713,440. The use of surfactants with polymeric hydrophilic polar groups, particularly polyethyleneglycol (PEG), such as Pluronics (Poloxamers) or PEGylated sorbitol or glycerol esters, with HLB values greater than about 8 or total PEG molecular weight greater than about 2,000 should preferably be minimized in the practice of this invention. Such surfactants are known to exhibit a “stacking effect” on surfaces; quasielastic light scattering measurements on particles dispersed with high-HLB Pluronics, for example, show an increase in particle diameter as the concentration of the high-HLB Pluronic increases, indicating the stacking of surfactant molecules at the particle surface, which could interfere with particle-cell interactions as discussed herein. Lipids chosen may, but need not, include lipids, fatty acids or oils (for example, Omega-3) which are components of or associated with eye tissue or tear film, or of prophylactic or therapeutic benefit to diseases and conditions of the eye.

It is useful for purposes of this disclosure to define the term “HLB”, for “Hydrophilic-hydrophobic balance”. With each surfactant is associated a number, generally between 0 and 20, which describes how hydrophilic the surfactant is—meaning of course, that it correlates with the aqueous solubility of the surfactant. In the original definition of HLB, the MW of the polar head was divided by the total MW of the surfactant molecule, and the resulting fraction multiplied by 20. Thus, a surfactant with an HLB of 10 has a polar head group that comprises 50% of the surfactant MW, to the extent that this definition is strictly maintained. In actual practice, assignment of the HLB for a particular surfactant is often done by the manufacturer, and often by running comparisons, with well-established surfactants of known HLB. But in any case, the following is universally agreed upon: highly polar surfactants with dominant head groups are assigned high HLBs greater than 10, while less polar surfactants with dominant hydrophobic groups (typically alkyl chains) are assigned low HLB values less than 10.

An important and universal rule that is very helpful in determining whether or not a lyotropic liquid crystal is of the reversed (or “Type II”) variety of utility in this invention, is that whenever a lamellar phase containing water, optionally oil, and one or more water-insoluble surfactants all of low HLB is convertible to a cubic or hexagonal phase by the reduction of water content, then the resulting cubic or hexagonal phase is of the reversed (Type II) variety. Contrariwise, the counterpart to this principle is also universally true, namely that whenever a lamellar phase containing water, optionally oil, and one or more water-soluble surfactants all of high HLB is convertible to a cubic or hexagonal phase by the addition of water, then the resulting cubic or hexagonal phase is of the normal (Type I) variety, and thus not a reversed liquid crystal of the instant invention. The importance of reversed (Type II) cubic and hexagonal phases over their normal (Type I) counterparts in the instant invention is in part a direct reflection of the relationship between insolubility and Type. That is, the insoluble Type II phases do not readily dissolve in relevant aqueous fluids such as the tear film fluid, intraocular fluid, blood, GI fluids, lymph, and cytoplasm but rather tend to retain their integrity in these fluids, which is obviously crucial in applications such as drug-delivery.

Polar solvents are required in the present invention for the creation of the lyotropic liquid crystalline phase material, and preferred as a continuous phase for dispersing said material. Usually, at least in the case of a bicontinuous cubic phase which is the preferred embodiment, the polar solvent composition in the liquid crystal and in the continuous (exterior) phase will ultimately be equal, or nearly equal, because the two are essentially in hydraulic continuity. The polar solvent may be: water; glycerol; formamide, N-methyl formamide, or dimethylformamide; ethylene glycol or other polyhydric alcohol; ethylammonium nitrate; other non-aqueous polar solvents such as N-methyl sydnone, N-methyl acetamide, dimethylacetamide, pyridinium chloride, etc.; or a mixture of two or more of the above, with water-glycerol being the most important of the mixtures. For the case of drug-delivery, the preferred polar solvents are water, glycerol, N,N-dimethylacetamide, and N-methylacetamide, as well as mixtures thereof. Water-glycerol mixtures are of extremely low-toxicity and are very compatible with many surfactants including phospholipids. Dimethylacetamide-glycerol mixtures are excellent for dissolving difficultly-soluble pharmaceutical compounds.

The incorporation of liquid, non-surfactant components with high partition coefficient (significantly hydrophobic) is very important in the practice of this invention, since such a compound can serve one or more of a number of roles: to co-solubilize actives and/or other useful compounds, and to modulate phase behavior, as well as in some cases to modulate interactions with biomembranes, act as oil-phase antioxidant, fluidize the bilayer, etc. Alpha-tocopherol and other preferred high-partition-coefficient diluents are described in U.S. Pat. No. 7,713,440.

### Applying the Invention

The uncoated particles of the present invention may be adapted to absorb one or more materials from the environment of the eye, or to release one or more materials, such as therapeutic agents. They can also be used, with or without an Active Pharmaceutical Ingredient (API), to lay down a lipid-rich coating on a tissue or liquid film of or associated with the eye, which coating, due to the bicontinuous channels comprising the reverse cubic and reverse hexagonal phase uncoated particles, allow flow of aqueous phase and its contents from one side of the coating, through the particles, to the other side. The aqueous pores would retain natural tears and water soluble compounds. The coating would not obstruct the circulation of aqueous or water soluble compounds from one side of the coating to the other, or between the interior of the particles and outside of the particles. Without wishing to be bound by theory, such a film, as established by either components of the particles or by the particles themselves (or fragments thereof), can apply in one or more areas or interfaces including, most preferably, the corneal surface, the mucin-rich region of the tear film, and/or the interface between the tear film and air. By lowering the surface tension of the tear film, and/or by improving the wettability of the corneal surface (for example, by deposition of polar lipid-rich material with polar moieties of the lipid establishing a more hydrophilic milieu), the integrity of the tear film can be improved and it's break-up time increased substantially. A substantial increase in tear-film break-up time would be an increase of about 50% or more, more preferably an increase of about 100% or more, and most preferably an increase of about 200% or more. Dwell time can be increased. Such particles protect the eye while allowing diffusion and delivery of water soluble actives, nutrients and other materials. In contrast with solution (i.e., non-particulate) formulations used in the treatment of dry eye or other ophthalmic conditions, such as the currently marketed formulation sold under the tradename “Endura”, the particulate formulations of the instant invention can significantly prolong the residence time of an active or drug compound in the eye, preferably prolonging this average residence time by at least a factor of two and more preferably by a factor of 10, and thereby improve it's therapeutic effect and/or absorption.

The nanometer-scale size achievable with particles of this invention allows them to enter into regions accessible only through small orifices, to imbibe into porous tissue structures, to absorb and release molecules with extremely short diffusion times, and to evenly and thoroughly coat surfaces and interfaces of interest. The charge associated with the particles enhances their interaction, including binding, with cells and bioactive compounds. With this in mind, particles of this invention can be applied through a wide range of ophthalmic routes: periocular, intraocular, conjunctival, subconjunctival, transconjunctival, peribulbar, retrobulbar, subtenons, transscleral, topical eye drop, topical gel, topical dispersion, intraorbital, intrascleral, intravitreal, subretinal, transretinal, choroidal, uveal, intracameral, transcorneal, intracorneal, intralenticular (including phakia and psuedophakia), and in or adjacent to the optic nerve. Systemic delivery of drug substances through the ocular route is also possible. The invention can be used, among other things, to aid in the use of contact lenses, or the conditions of the eye attendant to their use, and to treat dry eye, aqueous deficiency, mucin deficiency, meibomian gland dysfunction, neurogenic dry eye, and inflammation associated with dry eye and to protect and coat the ocular surface.

One very valuable aspect of the invention applies in particular to highly insoluble actives, insoluble drugs in particular. A major focus in drug development is the water solubility of drug candidates, and considerable resources are spent measuring, optimizing, and evaluating this solubility, even in cases where it is very low. The prevalent conception is, in fact, that this is a crucial parameter because, at some point in the path to absorption, the drug will have to dissolve in water en route to the target cell membrane. However, it is recognized in this invention that uncoated particles as disclosed herein, which interact intimately with target membranes, can greatly reduce or even circumvent the need for diffusion of “naked” drug (drug that is no longer in the particle core) across aqueous paths to reach the target membrane: a drug molecule dissolved in a particle of the present invention would be taken up directly from the particle into the cell membrane, and in some embodiments releasing in a sustained manner over an extended time.

Proteins, polypeptides, nucleic acids, polysaccharides, lectins, antibodies, receptors and other biomacromolecules are actives that can be particularly well suited for incorporation into particles and dispersions of the current invention. The tear film itself contains many different proteins, and delivery, sequestration or removal of proteins by means of the particles of this invention can have prophylactic and therapeutic effect. The lipid bilayer of the reversed liquid crystalline phase holds and protects them from degradation and deformation, and can provide the absorption enhancement properties discussed herein—which can be especially important in the case of macromolecules. In the case of nucleic acids in particular, but also in other cases, particles of the current invention could be of great utility in delivering actives to intracellular sites, such as the nucleus or nuclear membrane, the Golgi apparatus, the endoplasmic reticulum, the mitochondria, etc., and in such a case the transport-enhancing properties of the reversed liquid crystalline phase materials, particularly the reversed bicontinuous cubic phases, can be of high utility in the context of an uncoated particle. This is particularly useful for polyanionic drugs such as nucleic acids, which can be bound strongly to cationic particles of this invention.

In cases of pharmaceutical application of the invention where the drug is disposed in the particle in crystalline form (as opposed to solubilized), within a reversed liquid crystalline microparticle, and thus surrounded by a contiguous and continuous matrix of the liquid crystalline material, and particularly when the particles are also submicron in size, then the reversed liquid crystalline material portion of the particle can serve a number of functions simultaneously, including but not limited to: stabilizing the particles in dispersion; enhancing absorption by improving interactions with biomembranes and other barriers, including both plasma membranes and intracellular (e.g., nuclear) membranes; serving as a matrix for the solubilization of other excipients or co-factors; serving as a matrix for the solubilization of efflux protein inhibitors in particular; serving as a sustained release depot secondary to its mucoadhesion; providing a means by which to modulate, and even reverse, the effective charge on the drug; provide improved compatibility with certain drug formulation approaches; provide for modulation of the deposition characteristics of drugs by the presence of a bioadhesive and/or high-viscosity matrix; provide for poresize-selective protection from, or access of, biomacromolecules (e.g., albumin, proteases, nucleases, esterases) to the solid drug; in the case of a prodrug, provide for drug targeting or controlled release delivery by permselective access and/or controlled dissolution of the matrix; and provide for improved stabilization of the drug dispersion in biological fluids by the use of liquid crystals that have much lower solubilities than most of the surfactants previously used in nanocrystal stabilization.

Various other applications of microparticles in general are known, including those listed in U.S. Pat. No. 6,638,621, the complete contents of which are herein incorporated by reference.

Pharmaceutical compounds of use in treatments for the eye that are particularly well-suited for incorporation as active pharmaceutical ingredients in the instant invention include: cyclosporine, mycophenolic acids and its salts, mycophenolate mofetil and other immunosuppressants; triamcinolone; bupivacaine, lidocaine, procaine, tetracaine, mepivacaine, etidocaine, oxybuprocaine, cocaine, benzocaine, pramixinine, prilocaine, proparacaine, ropivicaines, chloroprocaine, dibucaine, and related local anesthetics; steroids and steroidal anti-inflammatory agents, including fluorometholone, prednisolone acetate, prednisolone phosphate and especially dexamethasone; antiinfectives such as bacitracin, erythromycin, polymyxin, neomycin, and tobramycin, ciprofloxacin, gentamycin, sulfacetamide, and combinations thereof. By incorporating, preferably by solubilizing in the reversed liquid crystalline phase, these or related active compounds into particles of this invention, and applying a dispersion containing such particles to the eye, efficacious delivery of the drug can be achieved by virtue of the electrostatic attraction between the cationic particle and anionic components of the eye, particularly the mucin layer, in combination with the ability of these particles to deliver the active to tissues particularly when the particles form a film in intimate contact with epithelial cells.

Other ophthalmic pharmaceutical actives which may be incorporated in the present inventions are: acetazolamide, amikaci, anecortave, antazoline, apraclonidine, atropine sulfate, azelastine, azithromycin, bacitracin, bacitracin zinc, betaxblol hydrochloride, bimatoprost, brimonidine, brinzolamide, bupivicaine, carpbachol, cartcolol hydrochloride, ceftazidime, ciprofloxacin hydrochloride, clindamycin, cromlyn, cyclopentolate hydrochloride, cyclosporine A, denufosol, dexamethasone, dexamethasone sodium phosphate, diclofenec sodium, dipivefrin hydrochloride, diquafosol, dorzolamide, doxycycine, edetate sodium, emadastine, epinastine hydrochloride, epinephrine, erythromycin, fludcinolone, 5 fuoruracil, fluoromethalone, fluoromethalone acetate, flurbiprofen sodium, fomivirsen, ganciclovir, gatifloxacin, gentimibin, gramicidin, imopenemn, ketotifin, ketrolac tromethamine; latanoprost, lerdelimumab, levocabastine, levofloxacin, levubunolol hydrochloride, lidocaine, lodoxamide, lotoprednol etabonate, medrysone, methazolamide, metipranolol, mitomycin, moxifloxacin, naphazoline, nedocromil, neomycin, ofloxacin, olopatadine, oxacillin, oxymetazoline hydrochloride, pegaptanib, pemirolast, pheniramine, phenylephrine hydrochloride, photofrin PIR 335, pilocarpine hydrochloride, polymixin B, prednisolone acetate, prednisolone sodium phosphate, proparaeaine, ranibizumab, rimexolone, scopolamine hydrobromide, sulfacetamide sodium, tetracaine, tetrahydrozoline hydrochloride, timolol, timolol maeate, tobramycin sulfate, travoprost, triamcinolone acetonide, trimethoprim, tropicamide, unoprostone, urea, vancomycin, and verteporfin. Also suitable are derivatives, analogs, and prodrugs, mixtures and combinations thereof.

In the area of pharmaceutics and nutriceutics, the particles of the present invention may be administered to a mammal (including a human), or other animal, by any of a variety of routes of administration which are well established and well known to those of skill in the art, in addition to the ophthalmic routes discussed elsewhere herein. These include but are not limited to oral (e.g., via pills, tablets, lozenges, capsules, troches, syrups and suspensions, and the like) and non-oral routes (e.g. parenteral, intravenous, intraperitoneal, intrathecal, intramuscular, subcutaneous, intra-arterial, rectal, intravaginal, sublingual, intraocular, transdermal, intranasal, via inhalation, in a suppository; and the like). The compositions of the present invention are particularly suited for topical admimstration, but, in some applications may be internally (e.g., IV, BP, subcutaneous, etc.) provided. The present invention is especially useful in applications where a difficultly soluble pharmaceutical active is to be delivered wherein said formulation is to be mixed with a water continuous medium such as serum, urine, blood, mucus, saliva, extracellular fluid, etc. In particular, an important useful aspect of many of the structured fluids of focus herein is that they lend themselves to formulation as water continuous vehicles, typically of low viscosity.

The microparticles of this invention have, by virtue of their richly amphophilic nature and their incorporation of high-partition coefficient liquid solvents and diluents (such as tocopherols and other tocotrienols, squalene, diacetylated monoglycides, linalool, benzyl benzoate, and strawberry aldehyde, all of which have octanol-water partition coefficients larger than about 1,000), are able to solubilize compounds that are of applicability in (e.g.) ophthalmics, and the adhesion to tissues of the eye can serve to increase corneal residence time and thus provide effective delivery of the compound to targeted tissues in the eye. This can be particularly effective in view of the strong microparticle-biomembrane interactions, for these uncoated reversed liquid crystalline phase particles, that can promote adsorption of the compound by cells of the eye and surrounding tissues.

Specific classes of compounds that can be incorporated and delivered include demulcents, emollients, lubricants, vasoconstrictors, antibiotics and antiseptics, antihistamines, immunosuppressants, local anesthetics, antiallergics, antifungals, vasoprotectants, anticoagulants, mucolytic and proteolytic compounds, antiglaucoma drugs, and antiinflammatories, anesthetics, anti-inflammatory, anti-helminthic, analgesics, steroids, non-steroidal inhibitors of the inflammatory cascade, anti-neoplastic, anti-angiogenic, calcineurin inhibitors, anti-ocular hypertensives, anti-virals, anti-bacterials, neuroprotectants, anti-apoptotics, medications for dry eye, pupil dilating medications (mydriatics and cycloplegics), ocular decongestants, anti-oxidents, photosensitizers, photodynamic therapy agents, mast cell stabilizers, monoclonal antibodies, quinolone antibiotics, intra-ocular pressure lowering agents. Demulcent and emollient active ingredients include: carboxymethycelluslose, glycerin, polysorbate 80, hydroxypropyl cellulose, hydroxypropyl methylcellulose, dextran 70, hypromellose, methycellulose, polyvinaly alcohol, PEG-5 to 800, proplylene glycol, white petrolatum, mineral oil, lanolin, hydroxypropyl guar, zinc sulfate, 0.25 percent; cellulose derivatives, including carboxymethylcellulose sodium, hydroxyethyl cellulose, hydroxypropyl methylcellulose and methylcellulose, dextran; gelatin; polyols liquids, including glycerin (glycerol), polyethylene glycol 300, polyethylene glycol 400, polysorbate 80 and propylene, glycol; polyvinyl alcohol; povidone; lanolin preparations, including anhydrous lanolin, lanolin; oleaginous ingredients, including light mineral oil, mineral oil, paraffin, petrolatum, white ointment, white petrolatum, white wax and yellow wax; sodium chloride, ephedrine hydrochloride, naphazoline hydrochloride, phenylephrine hydrochloride and tetrahydrozoline hydrochloride. All of these are identified in 21 CFR 349, Rev Apr. 1, 2005, Ophthalmic Drug Products for Over the Counter Use.

As is well known in the field, steroids are ubiquitous in ophthalmics and serve a number of pharmacological roles, and several of the Examples below detail the incorporation of steroids in microparticulate dispersions of the invention. Diagnostic compounds of importance in ophthalmics, such as dyes and radiopaque compounds, can also be incorporated, another case where adhesion to the eye and/or effective cellular delivery can be crucial. Nutrients of importance to the eye, such as in the prevention or treatment of macular degeneration, including zeaxanthin, carotenes, tocopherols, other vitamins, and especially combinations thereof, can also be delivered using this invention.

It must be stressed that for the treatment of dry eye and related conditions, administration of microparticles of this invention—with or without a pharmaceutical or ophthalmic active explicitly added—can be efficacious against the condition by virtue of the ability of the microparticles to lay down a film of lipid over the cornea and/or at the air-tear film interface. In other words, the lipid or lipids chosen to comprise the microparticles of the invention can be chosen from a wide array for various characteristics, and can themselves be active against the dry eye condition. Microparticles of the instant invention, in certain preferred embodiments, have phospholipids as a major component, preferably at least 25% concentration by weight and most preferably greater than or equal to about 30% by weight. Preferably, phosphatidylcholine (PC) comprises at least about 45% of the phospholipid in the microparticle, and most preferably comprises 70% or more of the phospholipid. Other lipids can be useful as components of microparticles of this invention, in part due to their role in helping to establish a longer-lived lipid film over the cornea or in association with the tear film, particularly when this results in a prolonged tear film break-up time. Preferred such lipids, which can in many cases be used in conjunction with phospholipids, are tocopherols, cholesterol, cholesterol esters, waxes, lanolin, mineral oil, paraffin, petrolatum, prostaglandins, leukotrienes, and glycolipids. Criteria for selecting lipids include their capacity to support microbial contamination. Certain lipids and related compounds are known to play a role in the health and disease of the eye, such that an excess or deficiency thereof is associated with a disease or conditions of the eye, and the lipidic component of the particles of the current invention may be selected for the purpose of adding or taking away such lipids from the environment of the eye, including eye tissues and the tear film

An important practical and economic aspect of the instant invention is the fact that many embodiments fall within, or at least substantially within, the guidelines of the FDA's monograph, 21CFR Part 349, for Ophthalmic Drug Products for Over-the-Counter Human Use. By using excipients, and in some cases actives, that are well-established in a regulatory sense as used in existing marketed ophthalmic products, the barriers to market entry are lowered. In several of the Examples below, a level of 0.2 to 1% of glycerol, polysorbate, propylene glycol, or PEG300/PEG400 is used so as to bring the formulation under the ranges specified in the monograph. Interestingly, in the case of many of the polymers listed in the monograph (typically as demulcents, in 21CFR349.12), the polymer can be incorporated into dispersions of the instant invention in two ways: it can be incorporated into the aqueous exterior phase (the continuous phase, outside the particles), or in the liquid crystalline particles themselves, as part of their composition. In the latter case, this is usually best accomplished by dissolving the polymer in the water that goes into forming the liquid crystal, prior to adding the surfactant or lipid(s) and any hydrophobes. It is important to point out that in the latter case, the overall loading of polymer can be very low even while it is substantial in the particles, so that interactions between polymer and components of the eye (such as mucins) can work synergistically with properties of the particles. Polymers that are of particular value in the application of this invention include carboxymethylcellulose and its salts, hydroxyethylcellulose, hypromellose, methylcellulose, dextran, gelatin and hydrolyzed gelatin, polyvinylalcohol, povidone, and polyethyleneglycols (PEGs, such as PEG400 and PEG300). Generally, higher MW polymers are retained longer in the pores of the reversed liquid crystalline phase particles of the invention than are low-MW polymers. Anionic polymers can show significant retention in particles of this invention, and can be used to bind to mucin-rich surfaces of the eye for improved corneal retention, for example, but in many (though not all) cases it will be important to ensure that the molar concentration of anionic charges in the polymer is less than the molar concentration of cationic charges in the particles, in order to maintain an overall cationic charge to the particle.

In the present invention it can be very effective to incorporate chemicals or chemical groups—often proteins or other biomacromolecules—that can be invoked to target particles temporally and spatially, for example, to target particles to specific sites in the body. Similarly, other bioactive compounds incorporated on or in the particles could serve important functions, such as: absorption enhancers such as menthol could be present so as to increase permeability of absorption barriers (lipid bilayers, gap junctions) prior to or concomitant with the release of drug; proteins or other adsorption-modulating materials could be incorporated that would inhibit unfavorable binding of endogenous proteins such as albumin; adjuvants could be incorporated that would enhance the effect of vaccine components or other immune modulating materials. Antibodies, steroids, hormones, oligo- or polysaccharides, nucleic acids, vitamins, immunogens, and even nanoprobes are all examples of a wide range of materials that could be attached to particles of the instant invention, either by solubilization or compartmentalization in the liquid crystalline material, or by covalent bonding, ionic bonding, coordinate bonding, hydrogen bonding, adsorption, specific biochemical interactions (such as avidin-biotin binding), or other chemical interactions with components in the particle. Similarly, antioxidants, preservatives, compounds to adjust osmolality and other compounds frequently used in formulating prescription and OTC products for human use may be incorporated.

For certain embodiments of the instant invention, including certain ophthalmic applications, it is preferable that the concentration of drug or active within the particle be at least about 0.3%, more preferably at least about 1%, and most preferably greater than or equal to about 3% on a wt/wt basis. This is important so that a therapeutic amount of drug be delivered in one or two drops of the formulation preferably, or less preferably 3-10 drops, preferably requiring administration at most once every 4 hours. Example 2 below provides a case in which the active, a local anesthetic in that case, has a concentration of about 3.5% in the reversed cubic phase.

The total charge and concentration of charged particles can be varied to vary the impact and effect of the particles on the treated tissue or associated compounds and on the condition being treated.

The following examples illustrate the present invention but are not to be construed as limiting the invention.

### Example 1

A reversed cubic liquid crystalline phase was prepared by thoroughly mixing 0.962 gm of propofol, 0.706 gm water, and 1.329 gm of soy phosphatidylcholine (from Avanti polar lipids). An amount 1.002 gm of this reversed bicontinuous cubic phase was dispersed in 20 ml of a solution of benzalkonium chloride 5. The average zeta potential was then measured (Beckman-Coulter DELSA instrument) and found to be about +74 mV.

### Example 2

The local anesthetic bupivacaine, in its free base form, and in the amount 0.176 gm, was combined with 0.700 gm linalool, 0.333 gm santalwood oil, 1.150 gm water, and 2.65 gm of the surfactant Pluronic L122. The resulting cubic phase is thus composed of excipients of very low toxicity; even santalwood oil has been shown to be of low toxicity by injectable routes (though it is not strictly speaking approved for use in injectable products). The cubic phase was then dispersed, using similar physical methods, using the cationic surfactant benzalkonium chloride. The resulting zeta potential distribution was centered around +55 mV, for the dispersion of charged-stabilized particles.

### Example 3

A cubic phase containing the therapeutic compound vitamin E was prepared by mixing 1.12 gm of vitamin E (alpha-tocopherol), 1.593 gm of soy phosphatidylcholine, and 0.788 gm of water. This was dispersed using benzalkonium chloride, and a zeta potential average of roughly +70 mV was recorded.

### Example 4

The same cubic phase as in the previous Example was stirred vigorous together with one-tenth its weight in sodium dantrolene, a skeletal muscle relaxant. This was then dispersed in aqueous benzalkonium chloride, with a 20:1 ratio of water to cubic phase, and a 0.06:1 ratio of surfactant to cubic phase. This was homogenized at high speed for 3 minutes. Zeta potential is particularly meaningful in this case, since the drug is anionic, whereas the dispersed cubic phase is cationic. Therefore, if “free” dantrolene is present then a peak will appear with a negative zeta potential, together with the peak from the cationic-stabilized particles, indicating that particles of this invention have not been produced. In fact, the strongly-colored (from the dantrolene sodium) dispersion was analyzed with the DELSA, and no peak was found at negative zeta potential. The analysis showed a single peak (at all four angles) centered at +72 mV. Thus, particles of the present invention were indeed produced, with nanosized crystals of the poorly-soluble skeletal muscle relaxant stabilized by their being embedded in a cationically-stabilized cubic phase particle of the current invention. An attempt to disperse dantrolene sodium with only the benzalkonium chloride, not using the cubic phase or any other liquid crystal, was made in order to evaluate the importance of the cubic phase in this Example. Thus, dantrolene sodium was dissolved in the aqueous phase at the same concentration, resulting in a 0.06:1 ratio of surfactant to dantrolene sodium, and the same homogenization protocol was applied. The DELSA measurement clearly shows a much smaller zeta potential than in the case where the cubic phase was used. This greatly increased charge in the case of the cubic phase particle is probably related to the much higher benzalkonium loading possible with the cubic phase (as expressed at the particle surface) as compared to the dantrolene sodium surface.

### Example 5

An amount of 1.0001 grams of the cubic phase identical to that from Example 13 in U.S. Pat. No. 7,713,440 were added to an equivalent 0.0638 gm of Benzalkonium Chloride (obtained from the Sigma Corporation) and 20.0 gm of distilled water as described in said the dispersion under the microscope. The zeta potential of the dispersion, taken at four different angles and run at 500 Hz for 180 seconds, averaged +47 mV which indicated a charge in the preferred range per the instant invention.

### Example 6

Soy phosphatidylcholine (lecithin 90G from Phospholipid GmbH), in the amount 12.52 grams, was combined Example 13. Benzalkonium chloride has been used in the injectable product Celestrone Soluspan. Following the methods described in said Example 13 of U.S. Pat. No. 7,713,440, the dispersion was homogenized, microfluidized, and analyzed under the microscope. The zeta potential for this dispersion was found to be +36 mV and thus indicated charge stability. It was run at 500 Hz for 180 seconds from four different scattering angles.

### Example 7

The same methods as were used for the preceding Example were used here to prepare a similar cubic phase except 0.9520 grams of Pluronic L-122 were used in place of the 0.9501 gm of Pluronic 123. An amount of 0.9989 gm of the cubic phase were then added to 0.0638 gm of benzalkonium chloride (obtained from the Sigma Corporation) and 20.0 gm of distilled water.

### Example 8

Methods similar to those utilized in Example 13 of U.S. Pat. No. 7,713,440 were used to microfluidize, homogenize, and analyze with 9.42 gm of deionized water, 0.99 gm of oleylamine, 3.54 gm of alpha-tocopherol (from Archer-Daniels-Midland), and 40 mg of rhodamine B base as dye, after which the mixture was heated to approximately 75° C. and stirred vigorously, resulting in a reversed bicontinuous cubic phase upon cooling to ambient temperature. In a 5-Liter container, 24.1 gm of mannitol, 8.00 gm of glycerol, 741 gm of deionized water, and 3.01 gm of 1M HCl were mixed to dissolve all solids. The reversed cubic phase, in the amount 21.77 gm, was then added to the aqueous solution, and the mixture homogenized with a Silverson high-speed homogenizer for 20 minutes on full speed. Alpha-tocopherol, in the amount 2.86 gm, was then added, and the entire mixture homogenized for another 20 minutes. This was then transferred to a Microfluidics 110L microfluidizer, and sheared under high pressure for 7.5 minutes. The dispersion was then filtered, first through a 5-micron pore size filter, then through a 1.2-micron syringe filter directly into crimp-seal vials. The vials were sparged with nitrogen gas, sealed with Teflon stoppers, and crimp-sealed. This formulation is referred to below as “LT-185”, and contains approximately 0.1% (1 mg/mL) oleylamine, most of which is present as the cationic form oleylamine hydrochloride. The pH of the dispersion was approximately 6.8. The average zeta potential was then measured on the DELSA and found to be +67 mV.

A similar formulation, named “LT-186”, was prepared in a nearly identical manner, but with 70.7 gm of cubic phase, said cubic phase being 5.65% oleylamine, thus resulting in an overall oleylamine concentration of approximately 0.5% (5 mg/mL oleylamine). The average zeta potential was then measured on the DELSA and found to be +79 mV.

Finally, a third formulation, named “LT-187”, was prepared in a similar manner, but with docusate sodium (“Aerosol-OT”) in place of oleylamine, at the level of 1.2 mg/mL overall, forming anionic particles. The average zeta potential was then measured on the DELSA and found to be −63 mV.

### Example 9

This Example used formulations LT-185, LT-186 and LT-187 prepared in Example 8.

Preparation of Mucin Slides. A 10 mg/ml concentration solution of porcine mucins in distilled water was first prepared. This solution was then applied to standard plain, pre-cleaned 75×25 mm glass slides. The slides were then dried in an oven at 50° C.

Mucin-binding testing. The following solutions were tested:

1. LT-185

2. LT-186

3. LT-187

4. Refresh Liquigel®

5. Soothe®

6. Systane®

7. Refresh Endura®

8. Genteal®

Composition of the controls, solutions 4 through 8:

Refresh Liquigel®: Carboxymethylcellulose 1%, boric acid, calcium chloride, magnesium chloride, potassium chloride, purified water, Purite (stabilized oxychloro complex). Sodium borate, sodium chloride.

Systane®: PEG 400 (0.4%), propylene glycol (0.3%), boric acid, calcium chloride, hydroxypropyl guar, polyquaternium-1, magnesium chloride, potassium chloride, purified water, sodium chloride, zinc chloride, (HCl and NaOH to adjust pH).

Soothe®: Light mineral oil 1%, mineral oil 4.5%, edetate disodium, octoxynol-40, polyhexamethylene biguanide, polysorbate 80, purified water, sodium chloride, sodium phosphate dibasic, sodium phosphate monobasic.

Refresh Ehdura®: Glycerin 1%, Polysorbate 80 1%, carbomer, castor oil, mannitol, purified water, sodium hydroxide.

Genteal®: Hydroxypropyl methycellulose, 0.3% Boric acid, GenAqua™ (sodium perborate), phosphonic acid, potassium chloride, purified water, and sodium chloride.

As stated above, all spiked solutions were spiked with rhodamine B base to approximately 0.1 mg/mL.

Two 50 microliter drops (representing the volume a typical eye drop bottle dispenses, noting that the active area of the slide was similar to that of the eye) using a pipette were placed in distinct locations on each mucin coated slide. The slides were rinsed in a saline, or water wash by being dipped and swirled around for two seconds, and the rinse was repeated.

Results: After the first several test materials were run, an informal comparison was done between the two rinsing solutions (water and 0.9% NaCl solution). All prototypes behaved similarly with the two rinsing solutions; 0.9% NaCl was then chosen because of its closer approximation of physiologic tears. Table 1 shows the details of each slide that was tested.

TABLE 1

| | | | Slide preparation |
|---|---|---|---|
| Slide number | Solution | Rinse | batch |
| 1 | 187 | Saline | A |
| 2 | 187 | Water | B |
| 3 | Genteal ® | Saline | A |
| 4 | Genteal ® | Water | B |
| 5 | Soothe ® | Saline | A |
| 6 | Soothe ® | Water | B |
| 7 | 186 | Saline | A |
| 8 | 186 | Water | B |
| 9 | Systane ® | Saline | A |
| 10 | Systane ® | Saline | B |
| 11 | Refresh Liquigel ® | Saline | A |
| 12 | Refresh Liquigel ® | Saline | B |
| 13 | Refresh Endura ® | Saline | A |
| 14 | Refresh Endura ® | Saline | B |
| 15 | 185 | Saline | A |
| 16 | 185 | Saline | B |
| E | 185, 186, 187 | Saline | C |
| F | 187, 185 | Saline | C |

Assessments of adhesion of the test article to the mucin coated slide: Observations of test slides under naked eye viewing conditions, and recorded by photograph, show several things:

1) The cationic “LyoCells” of the instant invention (slide #16) show strong binding of the dyed particles, rivaled only by the “Refresh®” product among the test articles;

2) Mucin adhesion of cationic Lyocells (185 and 186, slides #16 and 8) showed clear superiority compared with the other lipid-based dry eye products (Endura and Soothe, slides #14 and 6). As the lipid layer of the tears is thought to confer resistance to tear evaporation, it can be inferred that mucin binding should enhance clinical efficacy in a lipid based product.

3) The particles have substantially remained concentrated at the site where they were applied, demonstrating not only strong adhesion but also rapid kinetics of binding.

The concentrated spot areas on slide #16 were even more strongly and uniformly colored before a portion of the lower spot was stripped away during the wash; this material is more prone to stripping away than would otherwise be the case due simply to the greater thickness of the spot (in turn due to the rapid binding), which was dramatically seen in fluorescence microscopy, and in DIC mode.

Optical micrographs of selected slides were then performed as follows. Using low magnification (10× objective) in Differential Interference Contrast mode, photomicrographs were taken through a side port in a Reichert-Jung Polyvar microscope. All samples were photographed with the same microscope and camera conditions. These were imported into the image processing program PhotoImpact 7. The “Hue and Saturation” menu was invoked, and the Range of colors selected to cover the red and orange regions of the color spectrum, and the filter applied to each image; this was done to capture the dye over any other features in the micrographs, and to aid in converting to a grayscale image suitable for a patent disclosure. In this way, regions high in dye concentration appear strongly black, against a predominantly white or light-gray background. The photomicrographs so prepared included:

a photomicrograph of Slide #2, anionic LyoCell dispersion, in DIC mode. Dark areas are regions of high dye concentration.

a photomicrograph of Slide #6, Soothe, in DIC mode. Dark areas are regions of high dye concentration.

a photomicrograph of Slide #8, cationic LyoCell dispersion with 0.5% oleylamine, in DIC mode. Dark areas are regions of high dye concentration.

a photomicrograph of Slide #14, Endura, in DIC mode. Dark areas are regions of high dye concentration; and,

a photomicrograph of Slide #16, cationic LyoCell dispersion with 0.1% oleylamine, in DIC mode. Dark areas are regions of high dye concentration.

These micrographs clearly showed the strong binding of the cationic dispersion at 0.1% oleylamine. Indeed, careful inspection of the photos showed the deep thickness of the microparticle film, and this was dramatically evident in both DIC and fluorescence mode.

### Example 10

Didodecyldimethylammonium bromide (DDAB), a cationic double-chained surfactant, in the amount 0.143 gm, was admixed with 6.140 gm of oleylamine, 1.896 gm of Phospholipid 90G (Phospholipid GmbH), 0.778 gm of alpha-tocopherol (ADM), and 1.694 gm of water to make a reversed cubic phase. From this 2.999 gm were dispersed in 26.982 gm of water by first homogenizing for 15 minutes (Polytron 3000, speed 5), then microfluidizing for 3 cycles of 1.5 minutes each. The zeta potential measured (Beckman-Coulter DELSA instrument) was centered around +106 mV, at a pH of 7.16.

### Example 11

An amount 0.211 gm of oleylamine, 2.045 gm of Phospholipid 90G (Phospholipid GmbH), 0.768 gm of alpha-tocopherol (ADM), and 2.112 gm of water to make a reversed cubic phase, which was checked to be essentially free of birefringence in polarizing microscopy. From this 3.005 gm were dispersed in 27.003 gm of water by first homogenizing for 15 minutes (Polytron 3000, speed 6), then microfluidizing for 3 cycles of 1.5 minutes each. Eight mL was then filtered through a 0.45 micron poresize Millipore filter. The zeta potential measured (Beckman-Coulter DELSA instrument) was centered around +94 mV, at a pH of 7.47. Particle size (Beckman-Coulter N4) was unimodal, distributed nearly uniformly over a range of about 150 to 350 nm. Samples of this dispersion were made isotonic with glycerol, and with glycine, and found to be stable. It was also found that hydroxypropylcellulose (0.32%) and polyvinylpyrrolidone (PVP, 10,000 MW, 1.0%) could be added to the dispersion without causing flocculation.

### Example 12

The dispersion made in the previous Example was then tested in vivo for eye irritation. Healthy young adult albino rabbits (NZW (SPF)) of either sex from a single strain (NZW (SPF)) weighing 2 kg to 3 kg, was used for each test article or extract. The animals were selected from the stock colony following at least five days acclimation to the facility. These rabbits were free of ocular irritation prior to the test initiation. This determination was made using a slit lamp and fluorescein stain.

No longer than 24 hours before commencement of the test, rabbits were weighed and their eyes were examined for evidence of ocular abnormality by the McDonald-Shadduck Method using a slit lamp and fluorescein stain. A 0.1 ml volume of each extract test article was placed into the inferior ocular cul de sac (i.e., the cup formed by gently pulling the rabbits' lower right eyelid away from the eye) of each of three rabbits. The vehicle control was instilled into the left eye of each of these three rabbits. After application, the lids were held closed for approximately one (10 second. At 1, 24, 48 and 72 hours following instillation, both eyes of each rabbit were examined macroscopically for ocular irritation. After the 72 hour observation, the eyes were examined again by the McDonaid-Shadduck Method using a slit lamp and fluorescein stain. The cationic dispersion of the instant invention achieved a “pass” on this test, showing that it is non-irritating to the eye.

### Example 13

Cyclosporine, a drug that has utility in the treatment of dry eye and other conditions of the eye, in the amount 0.063 gm, was mixed with 0.242 gm of oleylamine, 2.748 gm of Phospholipid 90G (Phospholipid GmbH), 1.040 gm of alpha-tocopherol (ADM), and 2.037 gm of water, and 2 mg of rhodamine B base as a dye, to make a strongly-colored reversed cubic phase, which was checked to be essentially free of birefringence in polarizing microscopy. From this 4.599 gm were dispersed in 43.013 gm of a glycerol-mannitol solution (the glycerol being 1% and mannitol 2.6%, for tonicity), by homogenizing for 25 minutes (Polytron 3000, speed 17). In this dispersion, at least a portion of the cyclosporine is dispersed in the microparticles, rather than dissolved.

### Example 14

Cyclosporine was first solubilized at the level of 11% wt/wt in the acetylated monoglyceride product Acetem 90-50 (Danisco), with sonication to speed dissolution. This solution, in the amount 0.605 gm, was mixed with 0.167 gm of oleylamine, 1.298 gm of Phospholipid 90G (Phospholipid GmbH), 0.442 gm of alpha-tocopherol (ADM), and 1.180 gm of water. From this 2.48 gm were dispersed in an aqueous solution containing 1.032 gm of a glycerol, 3.003 gm of mannitol (for tonicity), 0.136 gm of monobasic potassium phosphate, and 93.3 gm of water, by homogenizing for 15 minutes (Polytron 3000, full speed). Alpha-tocopherol, in the amount 0.220 gm, was then added followed by 15 more minutes of homogenization. Two 30-mL vials were filled with about 20 mL of this cubic phase dispersion, and one of the vials was autoclaved at 121 C for 20 minutes. Both autoclaved and pre-autoclaved liquids were fine dispersions. Essentially all the cyclosporine in this dispersion is dissolved in the reversed cubic phase microparticles. The zeta potential measured (Beckman-Coulter DELSA instrument) was centered around +60 mV.

### Example 15

The steroidal drug triamcinolone acetonide was incorporated into particles of the invention at the level of approximately 1% in the particles, and approximately 1.5 mg/mL in the particle dispersion. In a 10 ml test tube, 1.0039 grams of sodium deoxycholate (Marcor) and 8.9971 grams of deionized water (Spectrum) were combined. The sample was vortexed until all deoxycholate was dissolved. In a 10 ml test tube, 0.0618 grams of triamcinolone acetonide (Spectrum), 1.4770 grams of deoxycholate solution, 1.5160 grams of benzyl alcohol (Sigma), 1.4663 grams of high-phosphatidylcholine lecithin (Phospholipid GmbH), and 1.4760 grams of Vitamin E (Archer Daniels Midland) were combined. The sample was vortexed and then slightly heated for 5 minutes. This mixture was referred to as a dissolved cubic phase, since the mixture in the absence of the benzyl alcohol solvent forms a reversed cubic phase. In a 100 ml beaker, 29.5083 grams of deionized water, 0.1534 grams of oleylamine (Aldrich), 0.0102 grams of Rhodamine B Base, and 4.8338 grams of dissolved cubic phase were combined. The sample was homogenized for 10 minutes, and then microfluidized for 8 runs of 1.5 minutes each. This resulted in a dispersion of particles of the invention (the cationic oleylamine is more than two-fold molar concentration the anionic deoxycholate, giving the particles a cationic charge) containing the triamcinolone. In a 150 mL beaker, 1.8011 grams of mannitol (Aldrich), 0.5928 grams of glycerol (Aldrich), and 57.6015 grams of deionized water were combined. Then in a 1000 MW dialysis tubing (Spectrum), 6.0073 grams of the dispersion were added. A stir bar was added to the dialysis bath and left to stir overnight. The next day, the sample remained a dispersion of particles of the invention, with the exterior aqueous phase containing mannitol and glycerol (used in a number of Examples herein for tonicity adjustment and emollient activity).

### Example 16

The Controlled Adverse Environment (“CAE”) model [or “Dry Eye Chamber”] is the premier model for conducting ophthalmic studies for dry eye treatments. The CAE is an environmental chamber that exacerbates ocular irritation and staining in a controlled manner by regulating temperature (mean 76° F., SD 6° F.), humidity (<10%), lighting conditions (adequate to illuminate the environment without causing photosensitivity), airflow (nonturbulent), and visual tasking (e.g., television, reading). This model, in a controlled manner, mimics the ocular surface effects, such as increased keratitis and increased ocular discomfort that are synonymous with being exposed to a challenging environment, such as during exposure to dry heat or forced hot air, working on a computer, prolonged visual tasking or the challenging environmental conditions experienced on an airplane with low humidity. Objective and subjective evaluations of ocular dryness can be made using various clinical end points, such as tear film break-up time (“TFBUT”), keratitis (corneal staining) and conjunctival staining, Ocular Protection Index (OPI), tear production, osmolality, and impression cytology, and subjective ocular discomfort analyses, allowing for the measurement of signs and symptoms according to standardized scales. Tear film break-up time measures the interval between the last complete blink and the first appearance of a micelle, defined as a random dry spot on the ocular surface. Patients with dry eye have an unstable tear film and therefore exhibit a rapid TFBUT, causing the ocular surface to be exposed to the drying effects of the external environment. This causes damage to the ocular surface, which is highlighted through the use of fluorescein dye and quantified as keratitis and conjunctival staining. Symptoms of dry eye are subjective, and each patient may use a different descriptive term to convey his or her ocular discomfort. Using a standardized ocular discomfort scale, the symptoms of dry eye can be evaluated during exposure to the CAE. Questions from the Ocular Surface Disease Index are used. Also of use are Schirmers test, tear film break up time, corneal staining with fluorescein, lissamine green, and/or Rose Bengal. New tear film imaging technologies (such as OCT) can be used to assess the impact of the eye drops on the tear film. (See, e.g., Ousler G W, Wilcox K A, Gupta G, Abelson M B, Annals of Allergy, Asthma, & Immunology 93(5):460-4, 2004 November, and, Mundorf T. Wilcox K A Ousler G W 3rd., Welch D, Abelson M B, Advances in Therapy 20(6):329-36, 2003 November-December).

The first study investigates the impact on ocular drying of LT-185, as prepared in Examples 8 and 9 above, on patients with normal ocular health exposed to a controlled adverse environment (CAE). Twenty individuals complete a randomized, double-masked, crossover study. Half, Group A, are controls and use a certain widely used OTC eye drop. The other half, Group B, receive LT-185. Participants are evaluated in the CAE. Baseline ophthalmic safety examinations are performed, including visual acuity (using the Early Treatment of Diabetic Retinopathy Study method) and slit-lamp biomicroscopy. Tear film break-up time measurements and keratitis and conjunctival staining evaluations are performed to quantify ocular dryness. Participant-reported ocular discomfort is recorded regularly during CAE challenge. For the TFBUT evaluations, 5 microL of nonpreserved 2% sodium fluorescein is instilled into the lower palpebral conjunctiva of each eye. Using a stopwatch, TFBUT is measured as the time from the last blink to the appearance of the first growing micelle. This procedure is repeated 3 times per eye to obtain the mean TFBUT. Keratitis (corneal staining) and conjunctival staining evaluations are performed using 5 microL of nonpreserved 2% sodium fluorescein instilled into the lower palpebral conjunctiva of each eye. The examiner waits 5 minutes after instillation to begin evaluation and grades the cornea (inferior, superior, and central) and conjunctiva (nasal and temporal) using a standardized scale from 0 to 4 points. Participants are exposed to a controlled adverse environment (CAE) for approximately 45 minutes, during which ocular discomfort (according to a standardized scale from 0 to 4 points) is evaluated every 5 minutes. Ophthalmic examinations are repeated immediately after CAE exposure. Nonparametric methods are implemented for analysis of staining and ocular discomfort data (Wilcoxon rank sum tests). Paired t-tests are used for TFBUT analysis. The average of both eyes is used in the analyses. Statistical significance is defined as P<05. Patients receiving LT-185 treatment demonstrate longer TFBUT and less keratitis and conjunctival staining.

The second study investigates the impact on ocular drying of LT-185, as prepared in Example 10 above, on patients known to have a diagnosis of dry eye, exposed to a controlled adverse environment (CAE). Patients (n=10 to 20) are in two groups, randomized to current therapy or LT-185 for two to four weeks, and crossover design. Patients are evaluated for objective ocular tolerance and subjective symptoms as in previous example. Patients receiving LT-185 treatment demonstrate longer TFBUT and less keratitis and conjunctival staining.

### Example 17

In a 20 mL scintillation vial, a supersaturated solution of gastric-mucin was prepared by dissolving 0.4983 gm gastric mucin (Spectrum) in 20.0119 gm of deionized water (Spectrum). The vial was vortexed for 1 minute and sonicated for one-half hour. The sample was separated into two 16 mL test tubes and centrifuged on a benchtop centrifuge for one-half hour, in order to remove undissolved or colloidal material. The supernatant was collected and filtered subsequently through 5 micron and 1.2 micron filters (GE Water & Process Technologies). In a 30 mL beaker, an oleylamine-containing cubic phase was prepared by stirring 2.5044 gm of phosphatidylcholine-rich lecithin (Phospholipon 90G from Phospholipid GmbH), 1.9189 gm deionized water (Spectrum), 0.1970 gm of oleylamine (Acros), and 1.4360 gm of vitamin E (Archer Daniels Midland Co.) for one-half hour. It should be noted that this cubic phase contained no mucins. Next, 1.483 gm of this cubic phase was smeared on sides of an 8 mL test tube. An amount 4.027 gm supersaturated gastric-mucin solution was added to the test tube and allowed to sit without agitation for 24 hours. The sample was prepared in duplicate with 1.525 gm oleylamine cubic phase and 4.036 gm of the gastric-mucin solution. Concurrently, 4.0099 gm supersaturated gastric-mucin solution was added to an 8 mL test tube and allowed to sit without agitation for 24 hours, as a control/comparison sample.

Respectively, 3.6053 g, 3.5184 g, and 3.8658 g supernatant (remaining gastric-mucin solution) from the test tubes were weighed onto watchglasses and evaporated in a 50° C. oven for 24 hours. Dried watchglasses were found to contain 0.0609 g, 0.0595 g, and 0.0796 g gastric-mucin. Therefore, the supersaturated gastric-mucin solution contained 2.06 % gastric-mucin and the cubic phase partitioning samples (those in contact with cubic phase) contained only 1.69% gastric-mucin suggesting that the gastric-mucin had diffused into the accessible aqueous pores of the cubic phase. Interestingly, since the amount of aqueous pore space in the cubic phase in contact with the 4 mL of mucin solution was only about 0.5 mL, this pore space volume was only one-eighth that of the mucin solution, and yet the mass of mucin outside the cubic phase was decreased by one-fourth due to infiltration of the cubic phase, meaning that the mucin partitioned preferentially into the cubic phase. Because the measured amount of mucin in the dried liquid outside the cubic phase could well be an overestimate, in case a small amount of cubic phase material leaked or dispersed into the exterior liquid, the preferential partitioning of mucins into the cubic phase could well be even stronger than the estimate calculated above.

### Example 18

The dispersion made according to the method of Example 11 was tested in vivo for eye irritation under a multiple dosing challenge. Healthy young adult albino rabbits (NZW (SPF)) of either sex from a single strain (NZW (SPF)) weighing 2 kg to 3 kg, was used for each test article or extract. The animals were selected from the stock colony following at least five days acclimation to the facility. These rabbits were free of ocular irritation prior to the test initiation. This determination was made using a slit lamp and fluorescein stain. No longer than 24 hours before commencement of the test, rabbits were weighed and their eyes were examined for evidence of ocular abnormality by the McDonald-Shadduck Method using a slit lamp and fluorescein stain. A 0.1 ml volume of each extract test article was placed into the inferior ocular cul de sac (i.e., the cup formed by gently pulling the rabbits' lower right eyelid away from the eye) of each of three rabbits. After application, the lids were held closed for approximately one (1) second. The right eye of each animal was dosed with 01.0 ml of test material once on Day 0, twice on Day 1 (approximately one hour apart), three times on Day 2 (approximately one hour apart), and four times on Day 3 (approximately one hour apart.) The left eyes were left untreated. Macroscopic evaluation was performed prior to initiation and approximately seven (7) hours post the firs instillation each day. Slit lamp evaluation as performed prior to initiation and approximately seven (7) hours post the first instillation on Day 3. The test article, when administered in multiple instillations in this manner, is not considered an eye irritant in the Ocular Irritation Test.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of treatment of a disease or condition of the eye in a human or other animal subject comprising the step of providing to an eye of a subject a composition comprising a stabilized dispersion of uncoated cationically charged particles of reversed cubic or reversed hexagonal phase lyotropic liquid crystalline material said particles having an average diameter ranging from 10 nanometers to 100 microns.

2. The method of claim 1 wherein said disease or condition is dry eye.

3. The method of claim 1 wherein said composition further comprises an active pharmaceutical ingredient.

4. The method of claim 1 wherein said particles further comprise an active.

5. The method of claim 4 wherein said active is dissolved or dispersed or solubilized or otherwise incorporated within said reversed cubic phase or reversed hexagonal phase material.

6. The method of claim 4 wherein said active is selected from the group consisting of cyclosporine; mycophenolic acids and its salts, mycophenolate mofetil and other immunosuppressants; triamcinolone; bupivacaine, lidocaine, procaine, tetracaine, mepivacaine, etidocaine, oxybuprocaine, cocaine, benzbcaine, pramixinine, prilocaine, proparacaine, ropivicaines, chloroprocaine, dibucaine, and related local anesthetics; steroids and steroidal anti-inflammatory agents, including fluorometholone, prednisolone acetate, prednisolone phosphate and especially dexamethasone; antiinfectives such as bacitracin, erythromycin, polymyxin, neomycin, and tobramycin, ciprofloxacin, gentamycin, sulfacetamide, and combinations thereof.

7. The method of claim 4 wherein said active is selected from the group consisting of: demulcents, emollients, lubricants, wetting agents, vasoconstrictors, antibiotics and antiseptics, antihistamines, immunosuppressants, local anesthetics, antiallergics, antifungals, vasoprotectants, anticoagulants, mucolytic and proteolytic compounds, antiglaucoma drugs, and antiinflammatories, anesthetics, antiinflammatories, anti-helminthics, analgesics, steroids, non-steroidal inhibitors of the inflammatory cascade, anti-neoplastics, anti-angiogenics, calcineurin inhibitors, anti-ocular hypertensives, anti-virals, anti-bacterials, neuroprotectants, anti-apoptotics, medications for dry eye, pupil dilating medications (mydriatics and cycloplegics), ocular decongestants, anti-oxidents, photosensitizers, photodynamic therapy agents, mast cell stabilizers, monoclonal antibodies, quinolone antibiotics and intra-ocular pressure lowering agents.

8. The method of claim 1 wherein said providing step is performed by a route of administration to the eye selected from the group consisting of: periocular, intraocular, conjunctival, subconjunctival, transconjunctival, peribulbar, retrobulbar, subtenons, transscleral, topical eye drop, topical gel, topical dispersion, intraorbital, intrascleral, intravitreal, subretinal, transretinal, choroidal, uveal, intracameral, transcortical, intracorneal, intralenticular (including phakia and psuedophakia), and in or adjacent to the optic nerve.

9. The method of claim 8 wherein said route of administration is topical eye drop.

10. The method of claim 1 wherein the particles bind to the mucin layer of the eye.

11. The method of claim 1 wherein said providing step forms a continuous layer over all or a portion of the surface of the eye tissue, including the corneal tissue.

12. The method of claim 1 wherein said providing step forms a continuous layer over all or a portion of the tear film at the tear film—air interface.

13. The method of claim 1 wherein said reversed cubic or reversed hexagonal phase material adheres to mucins or to mucosal tissue.

14. The method of claim 1 wherein said reversed cubic or reversed hexagonal phase material adheres to anionically charged cells or tissues.

15. The method of claim 1 further comprising the step of preventing or retarding break up or evaporation of the tear film of the eye.

16. The method of claim 1 further comprising the step of absorbing or adsorbing one or more materials from an environment of the eye with said composition.

17. The method of claim 1 in which said composition comprises continuous aqueous channels such that said composition when provided to the tear film of the eye, permits the circulation of aqueous phase, including tears and water soluble compoundslytes, through, throughout and across said particles.

18. The method of claim 1 wherein the providing step lowers surface tension of a tear film of said eye.

19. The method of claim 1 wherein the providing step increases wettability of a corneal surface of said eye.

20. The method of claim 1 wherein the providing step reduces evaporation of a tear film of said eye.

21. The method of claim 1 wherein said composition prolongs corneal residence time.

22. The method of claim 1 wherein said composition increases dwell time.

23. The method of claim 1 wherein said composition enhances lipid coating or takes the place of lipid coating on said eye.

24. A composition for the treatment of diseases or conditions of the eye in a human or other animal comprising a stabilized dispersion of uncoated charged particles, or a plurality of uncoated charged particles without a carrier fluid which will be stabilized in dispersion as uncoated charged particles upon the addition of said carrier fluid, wherein said uncoated charged particles are formed from reversed cubic or reversed hexagonal phase lyotropic liquid crystalline material, said particles having an average diameter ranging from 10 nanometers to 100 micron.

25. The compositions of claim 24 wherein said particles adhere to mucins or mucosal tissue.

26. The composition of claim 24 wherein a charged compound imparts a cationic charge on the composition.

27. The composition of claim 26 wherein the charged compound is oleylamine or stearylamine or benzalkonium chloride.

28. The composition of claim 24 wherein a charged compound imparts an anionic charge on the composition.

29. The composition of claim 24 wherein the composition, when applied to the surface of the eye, is non-irritating.

30. The composition of claim 24 wherein the uncoated charged particles include phosphatidylcholine and vitamin E.

31. The composition of claim 24 wherein said composition includes said plurality of uncoated charged particles without said carrier fluid, and wherein said carrier fluid which forms said stabilized dispersion is water or an aqueous medium.

32. The composition of claim 30 wherein said aqueous medium is selected from the group consisting of natural or artificial tears.

33. A composition for the treatment of diseases or conditions of the eye in a human or other animal comprising a matrix material comprising continuous aqueous channels permeating the material, wherein said composition, when administered to the tear film of the eye, permits the circulation of aqueous phase, including tears, and water soluble compounds, including but not limited to active pharmaceutical agents, actives, nutrients, proteins, and electrolytes, through, throughout and across said material, including said particles.

34. A method of treatment of a disease or condition of the eye in a human or other animal subject comprising the step of providing to an eye of a subject a composition comprising a stabilized dispersion of uncoated charged particles of reversed cubic or reversed hexagonal phase lyotropic liquid crystalline material said particles having an average diameter ranging from 10 nanometers to 100 microns.

35. The method claim 34 wherein said uncoated charged particles have an anionic charge.

36. The method of claim 34 wherein said uncoated charged particles have a cationic charge.

37. A method of treatment of a disease or condition of the eye in a human or other animal subject comprising providing to an eye of a subject a matrix material comprising continuous aqueous channels permeating the material, wherein said composition, when administered to the tear film of the eye, permits the circulation of aqueous phase, including tears, and water soluble compounds, including but not limited to active pharmaceutical agents, actives, nutrients, proteins and electrolytes, through, throughout and across said material, including said particles.
